(19) United States
(12) Patent Application Publication
(10) Pub. No.: US 2005/0171177 A1
(43) Pub. Date: Aug. 4, 2005

(54) Method of treatment of Candida isolates

(76) Inventors: Daniel J. Thompson, Ballwin, MO (US); Elio P. Mariani, Chesterfield, MO (US); R. Saul Levinson, Chesterfield, MO (US); Herbert G. Luther, Chesterfield, MO (US)

Correspondence Address: NATH & ASSOCIATES, 1030 15th STREET, NW, 6TH FLOOR, WASHINGTON, DC, 20005, US

(21) Appl. No.: 10/767,431

(22) Filed: Jan. 30, 2004

(51) Int. Cl. A61K 31/4178; A61K 31/22; A61K 31/01

(52) U.S. Cl. 514/397; 514/546; 514/762

(57) ABSTRACT

The present invention relates to methods of treatment vulvovaginitis caused by nonspecified Candida isolates using controlled release antimycotic delivery systems. The invention utilizes a minimal number of administrations to obtain cessation of the condition. The methods and systems are especially effective against any Candida species causing vaginal irritation, and thus reduce the need for identification of the isolate prior to treatment.

### BACKGROUND OF INVENTION

1. Field of the Invention

The present invention is directed to methods of treatment of a nonspecified Candida species isolate using antimycotic delivery systems. These systems are suitable for use in the vaginal cavity. The invention is additionally concerned with methods utilizing preparations demonstrating a controlled, extended or sustained release of the active and/or therapeutic agent and a minimal number of administrations to produce efficacy upon administration of said delivery system. The methods and systems are especially effective against Candida species causing vaginal irritation, and thus reduce the need for identification of the isolate prior to treatment.

2. Description of the Related Art

The management of the female reproductive system for the prevention, treatment, mitigation, diagnosis and cure of diseases and the prevention of conception typically involves diagnosis of the specific condition and administration of an active agent or agents to the vaginal cavity and its environs. Regarding the diagnosis and treatment of vulvovaginal candidiasis, the currently employed delivery systems, regardless of formulation or method of manufacture, have not reliably demonstrated the ability to treat vaginal candidiasis conditions regardless of the isolate. Prior identification of the specific species inducing the symptoms is required in order to assure administration of the appropriate etiologic agent. This may be attributed to both the active agent or agents and the capabilities of the delivery system used for administration.

Vulvovaginitis is a common disorder that can affect females of all ages. Vulvovaginitis encompasses a variety of disorders characterized by inflammation that may be secondary to multiple causes, including infection, irritation, allergy, and systemic disease. Etiologies and approach to a patient with vulvovaginitis are age dependent. This inflammatory condition occurring in the lower female genital tract may be secondary to bacterial overgrowth, i.e., bacterial vaginosis. However, the pathology is not one of inflammation on histologic specimen. Pathophysiology of vulvovaginitis depends on the etiology.

Vulvovaginitis in women of childbearing age usually is caused by at least one of the following: bacterial vaginosis, Trichomonas species, and/or Candida species. Each of these can be found in the vagina of asymptomatic women, although no studies clearly elucidate why they sometimes produce clinical symptoms. Vaginal candidiasis, or vulvovaginal candidiasis, commonly is caused by Candida albicans and occasionally by Candida glabrata or Candida tropicalis. This organism is found in the vagina of 25% of asymptomatic women. Infection occurs with the overgrowth of Candida species, possibly triggered by broad-spectrum antibiotics or other factors influencing the vaginal milieu. Pregnancy predisposes women to infection because high hormonal levels and increased vaginal glycogen content favor growth of Candida species. Underlying medical conditions, such as diabetes mellitus, hypothyroidism, or human immunodeficiency virus (HIV), also predispose patients to candidal infections. Candida species are not sexually transmitted and usually are not associated with other gynecologic infections. Thus, vaginal candidiasis is a common etiology, especially in tropical climates. It usually is considered slightly less common than bacterial vaginosis, yet approximately three fourths of women experience at least one bout of candidal vulvovaginitis. Further, a small percentage of women who are treated successfully for an initial episode of candidal vulvovaginitis develop chronic or recurrent candidal infection. Although many do not, some women have predisposing factors such as diabetes mellitus, oral contraceptive (OCP) or antibiotic use, or immunodeficiency, or they wear tight-fitting undergarments. Treatment of this subpopulation of women may be challenging.

The vaginal cavity is subject to conditions rendering it a target for disease and infection, which increases the criticality of the fact that administration of active pharmaceutical agents to the vaginal cavity is challenging. Physiologically, it is extremely difficult to deliver an active agent to this area for an extended period of time. Further, the vaginal cavity exhibits an aqueous environment containing secreting glands whose fluids create an acidic pH in the range of 4.5 to 5.5. The environment of the vagina is conducive to the growth of various microbes, such as bacteria, fungi, yeast and other microorganisms which cause vulvovaginal conditions since it is warm, moist and dark. It is also the vestibule for menstrual debris and the residual seminal fluid from sexual intercourse. The crevices of the vaginal cavity facilitate the retention of undesirable bacteria, fungi, yeast and other microorganisms, as well as the debris from menstruation and sexual intercourse. The vaginal cavity is also subject to considerable physical deformation, such as during sexual intercourse or during the insertion of tampons.

Active agents having pharmaceutical qualities have been developed and approved for use in the treatment of conditions and diseases of the vaginal cavity and the prevention of conception. These include fungicides, antibiotics, spermicides, etc. Although pharmaceutically active agents have been developed, it has been difficult to achieve optimal potential effectiveness from these agents due to the inadequacy of currently available drug delivery systems. In this regard, it should be noted that no approved or suitable system, which releases the pharmaceutically active agent for three hours or more, has shown efficacy for use in the treatment of a vulvovaginal candidiasis condition when determination of the isolate is impossible or impractical.

Known systems exhibit limited effectiveness in situations where identification of the species of microorganism is impossible or impractical. Such is often the case for women of child bearing age and those of postmenopausal age. A substantial time investment is required to identify an isolate using known laboratory methods, such as a KOH smear test. In the interim, the condition remains untreated and symptoms worsen. Diagnosis confirmation requirements typically associated with known vulvovaginal treatments serve to identify the specific microorganism causing the condition. However, identification is not limited to one particular test. While the KOH smear test is common, separate laboratory procedures are required to rule out other pathogens. Thus using the methods of the known treatments may require a smear and a number of cultures. A typical culture procedure may take 48 to 72 hours and beyond, and start of treatment is thereby further extended.

Further, even once the genus and species is determined and treatment ensues, known delivery systems fail to provide appropriate relief due to rapid release of the active agents in an uncontrolled manner. Conventional systems also result in a relatively high systemic absorption of the active agent, which may be due in part to the instability of the system. A controlled release system delivers the active agent to the site of action, absorption or use in a predetermined manner. This contrasts with conventional immediate release systems, which require frequent repetitive dosing in order to achieve the desired level of active agent. An unexpected advantage of a controlled release system is that the drug is administered fewer times a day than conventional systems since the drug level in the vaginal cavity is maintained at a constant level. Unfortunately, the controlled release systems of the prior art do not affect the total number of days that are required to treat a condition.

The present invention is advantageous because it provides a method of treatment of a vulvovaginal candidiasis condition wherein the specific isolate does not require identification. The method utilizes a delivery system to administer an active agent in a controlled manner in the vaginal cavity for an extended period of at least several days. The vaginal drug delivery system may take the form of a multi-phase liquid or semi-solid, which is easily introduced into the vaginal cavity. Additionally, due to the bioadhesive nature of the delivery system, the material introduced into the vaginal cavity does not seep from this body cavity in an offensive manner. In comparison to conventional vaginal creams and ointments, the present technology is further advantageous in that it reduces the number of administrations needed to obtain efficacy. Thus, the present technology requires no predetermination of the Candida species prior to administration and needs to be administered only once to affect the same cure. In this manner, overall treatment time is greatly and unexpectedly reduced.

A further advantage of the present technology is the substantial cost reduction for effective treatment of vaginal infections by eliminating the need for a pretreatment determination of the Cadida species and providing treatment in a single dose.

### SUMMARY OF THE INVENTION

The present inventive subject matter is directed to A method for the local treatment of a vulvovaginal candidiasis condition diagnosable by a KOH smear test or other fungal speciation test, which comprises: treating said vulvovaginal candidiasis condition caused by a species of Candida selected from the group consisting of dubliniensis, tropicalis, glabrata, parapsilosis, krusei, and lusitaniae by applying to the vaginal tissue of a human a formulation comprising about 35 to about 45% w/w sorbitol solution; about 3 to about 8% w/w propylene glycol; about 0.001 to about 1% w/w edetate disodium; about 5 to about 11% w/w mineral oil; about 0.5 to about 5% w/w polyglyceryl-3-oleate; about 0.5 to about 5% w/w glyceryl monoisostearate; about 0.001 to about 1% w/w microcrystalline wax; about 0.5 to about 2% w/w silicon dioxide; about 0.001 to about 1% w/w methylparaben; about 0.001 to about 1% w/w propylparaben; about 25 to about 45% w/w water; and about 0.5 to about 5% w/w butoconazole nitrate; and wherein the treatment is a single dose treatment.

The present inventive subject matter is further drawn to a method for the treatment of a vaginal fungal condition, which comprises: administering a single dose composition comprising about 38 to about 40% w/w sorbitol solution; about 4 to about 6% w/w propylene glycol; about 0.01 to about 0.5% w/w edetate disodium; about 6 to about 9% w/w mineral oil; about 2 to about 3% w/w polyglyceryl-3-oleate; about 2 to about 3% w/w glyceryl monoisostearate; about 0.01 to about 0.8% w/w microcrystalline wax; about 0.09 to about 0.9% w/w silicon dioxide; about 0.01 to about 0.5% w/w methylparaben; about 0.01 to about 0.5% w/w propylparaben; about 30 to about 40% w/w water; and about 1.5 to about 3.5% w/w butoconazole nitrate; wherein the vaginal fungal condition is a vulvovaginal candidiasis condition caused by a Candida species selected from the group consisting of dubliniensis, tropicalis, glabrata, parapsilosis, krusei, and lusitaniae, and wherein the ratio of polyglyveryl-3-oleate to glyceryl monoisostearate is about 1:0.1-10.

Still further, the present inventive subject matter is drawn to a method for the treatment of an unidentified vulvovaginal fungal condition, which comprises: administration to said fungal condition a bioadhesive, single dose treatment formulation comprising from about 0.500 to about 5.000% w/w butoconazole nitrate; and wherein the unidentified vulvovaginal fungal condition is caused by a Candida species selected from the group consisting of dubliniensis, tropicalis, glabrata, parapsilosis, krusei, and lusitaniae.

Yet further, the present inventive subject matter is drawn to a method for the treatment of a fungal condition diagnosable by KOH smear test or other fungal speciation test, which comprises: application to a vulvovaginal candidiasis condition caused by a member selected from the group consisting of Candida dubliniensis, Candida tropicalis, Candida glabrata, Candida parapsilosis, mycelial Candida, Candida krusei, and Candida lusitaniae and mixtures thereof of a treatment comprising: about 35 to about 45% w/w sorbitol solution; about 3 to about 8% w/w propylene glycol; about 0.001 to about 1% w/w edetate disodium; about 5 to about 11% w/w mineral oil; about 0.5 to about 5% w/w polyglyceryl-3-oleate; about 0.5 to about 5% w/w glyceryl monoisostearate; about 0.001 to about 1% w/w microcrystalline wax; about 0.5 to about 2% w/w silicon dioxide; about 0.001 to about 1% w/w methylparaben; about 0.001 to about 1% w/w propylparaben; about 25 to about 45% w/w water; and about 0.5 to about 5% w/w butoconazole nitrate.

The present inventive subject matter is further drawn to a method for the local treatment of an unidentified vaginal fungal condition comprising: a single administration of a composition consisting essentially of: about 38 to about 40% w/w sorbitol solution; about 4 to about 6% w/w propylene glycol; about 0.01 to about 0.5% w/w edetate disodium; about 6 to about 9% w/w mineral oil; about 2 to about 3% w/w polyglyceryl-3-oleate; about 2 to about 3% w/w glyceryl monoisostearate; about 0.01 to about 0.8% w/w microcrystalline wax; about 0.09 to about 0.9% w/w silicon dioxide; about 0.01 to about 0.5% w/w methylparaben; about 0.01 to about 0.5% w/w propylparaben; about 30 to about 40% w/w water; and about 1.5 to about 3.5% w/w butoconazole nitrate.; and wherein the administration is to a vulvovaginal candidiasis condition caused by any member selected from the group consisting of dubliniensis, tropicalis, glabrata, parapsilosis, krusei, and lusitaniae.

Another embodiment of the present inventive subject matter is a method for the treatment of a fungal condition diagnosable by KOH smear test or other fungal speciation, comprising: treating a candidiasis condition caused by a species selected from the group consisting of dubliniensis, tropicalis, glabrata, parapsilosis, krusei, and lusitaniae by applying to the vaginal tissue a multiphase formulation in a single dose; wherein the multiphase formulation comprises: a hydrophilic phase, which comprises: about 38 to about 40% w/w sorbitol solution; about 3 to about 8% w/w propylene glycol; about 0.001 to about 1% w/w edetate disodium; about 25 to about 45% w/w water; and about 0.5 to about 5% w/w butoconazole nitrate; and a hydrophobic phase which comprises about 5 to about 11% w/w mineral oil; about 0.5 to about 5% w/w polyglyceryl-3-oleate; about 0.5 to about 5% w/w glyceryl monoisostearate; about 0.001 to about 1% w/w microcrystalline wax; about 0.5 to about 2% w/w silicon dioxide; about 0.001 to about 1.000% w/w methylparaben; and about 0.001 to about 1% w/w propylparaben.

A further embodiment of the present inventive subject matter is drawn to a method for the treatment of an undiagnosable vulvovaginatis condition comprising: treating a condition caused by a species of Candida selected from the group consisting of dubliniensis, tropicalis, glabrata, parapsilosis, krusei, and lusitaniae by applying to the vaginal tissue a multiphase formulation in a single dose to provide a Candida species kill rate of about 50 to about 100% for a period of at least about 4 days.

### DESCRIPTION OF THE PREFERRED EMBODIMENTS

The purpose of the present invention is to provide methods of treatment for vulvovaginal candidis, which do not require determination of the Candida species prior to initiation of treatment, utilizing a vaginal delivery system. The systems are characterized by their ability to deliver agents to a specific site, the vaginal cavity, in a controlled manner over a prolonged period of time. The systems are bioadherent to the epithelial tissue and are comprised of at least two phases. The systems, when in the vaginal environment, retain their integrity and display physical stability for an extended residence time within the vaginal cavity.

As discussed above, the vaginal cavity produces an aqueous environment conducive to the growth of bacteria, fungi, yeast and microorganisms. The systems of the prior art are not optimally effective for treating such conditions either due to their water miscibility, lack of bioadhesion, or lack of physical stability in the vaginal environment of 37 degrees C. The vaginal cavity as defined herein not only includes the vagina, but also associated surfaces of the female urinary tract, such as, the ostium of the urethra. Delivery systems are a combination of nonactive ingredients which serve to solubilize, suspend, thicken, dilute, emulsify, stabilize, preserve, protect, color, flavor and fashion an active agent into an acceptable and efficacious preparation for the safe and convenient delivery of an accurate dose of said active agent.

The term “active agent” as used herein refers to agents selected from the group consisting of antifungal agents, antibacterial agents, antimicrobial agents, antiviral agents, spermicides, hormone agents, growth enhancing agents, cytokines, antitrichomonial agents, antiprotozoan agents, antimycoplasm agents, antiretroviral agents, nucleoside analogues, reverse transcriptase inhibitors, protease inhibitors, contraceptive agents, sulfadrugs, sulfonamides, sulfones, hygiene agents, probiotic agents, vaccine agents, antibody agents, peptide agents, protein agents, polysaccharide agents, nucleic acids, plasmids, liposomes, carbohydrate polymers, transgenic bacteria, yeast, chemotherapeutic agents, steroid agents, growth enhancing agents, libido enhancers, androgenic substances, chitin derivatives, environment modifying agents such as pH modifiers, and mixtures and combinations thereof. Preferable antimicrobial agents are selected from the group consisting of butoconazole, butoconazole nitrate, salts thereof, complexes of butoconazole base and mixtures thereof.

It is essential to the present inventive formulations that the delivery system not only release an active agent, but that it releases the agent in a controlled manner to a site of optimal absorption or action. That is, an agent is made available for absorption, pharmacological or other effect at a site of absorption or action, in an amount sufficient to cause a desired response consistent with the intrinsic properties of the agent and which provides for maintenance of this response at an appropriate level for a desired period of time. Thus, the systems described herein are characterized by the controlled release of an active substance from a delivery system at a receptor site, site of action, site of absorption, or site of use and the achievement of the desired effect at that site. The systems of the invention are not miscible in water and are not harmful for use in the vaginal cavity.

Of note in the present system is the fact that long term, controlled and/or sustained release can be affected over a long period of time, at least about 24 hours to about 96 hours and as long as 7 days, through the administration of a low number of doses. In some cases as little as one dose can be administered to cover a treatment period of a number of days. Doses given once daily, multiple daily doses, every other day, every two, three, four days, etc., are within the scope of this invention. Alternatively, for treating recurring conditions, administration on the first and fourth days are feasible.

Not only does the present system have the ability to deliver an active pharmaceutical ingredient, i.e., an active agent, over an extended period of time, but the active also retains a relatively low plasma concentration (C max) throughout the administration.

Also of note in present systems is the ratio of emulsifiers in the oil phase. The preferred ratio of emulsifiers is between about 1:0.1-10. More preferably, the ratio of emulsifiers is between about 1:0.5-2. Emulsifiers for use in the present systems include polyglyveryl-3-oleate, glyceryl monoisostearate, and [please add additional emulsifiers]. Without being limiting in theory, it is believed that this ratio of emulsifiers imparts additional stability to the systems.

The systems are comprised of unit cells. These unit cells are the basic, nondivisible, repeating units of the system. The unit cells have internal and external phases, which represent the internal and external phases of the systems. The systems may be described in conventional classifications, such as emulsions, emulsions/dispersions, double emulsions, suspensions within emulsions, suppositories, foams, etc. The systems are usually in the form of emulsions either of medium or high internal phase ratio, preferably greater than 70% and more preferably greater than 75% by volume. The delivery systems are liquids or semi-solids with viscosities that range from 5,000 to one million centipoise, preferably 350,000 to 550,000 centipoise. The systems in order to adhere to the vaginal cavity must have sufficient viscosity to retain their integrity.

Given the new and improved formulations for administering an active agent, butoconazole can be used not only as an antimicrobial agent, but also as an antifungal agent.

The internal phase of the unit cells may be discontinuous and is nonlipoidal. The nonlipoidal character renders the internal phase miscible with water. Preferably the internal phase comprises water, glycerine, or combinations thereof. Generally, it is desirable that the internal phase be of high osmotic pressure. The internal phase may be multiphasic and may be a solution, suspension, emulsion or combination thereof and it contains at least a portion of the active agent. Also, the internal phase may contain suspended solids, emulsions, osmotic enhancers, extenders and dilutants, as well as fragrances, colors, flavors, osmostic agents and/or buffers.

The resistance of a solution to changes in hydrogen ion concentration upon the addition of small amounts of acid or alkali is termed buffer action. A solution possessing such properties is known as a buffer solution. It is said to possess reserve acidity and reserve alkalinity. Buffer solutions usually consist of solutions containing a mixture of a weak acid and its sodium or potassium salt or of a weak base and its salt. A buffer then is usually a mixture of an acid and its conjugate base.

The solution containing equal concentrations of an acid and its salt, or a half-neutralized solution of the acid, has maximum buffer capacity. Other mixtures known in the art also possess considerable buffer capacity, but the pH will differ slightly from the half-neutralized acid.

The preparation of a buffer solution of a definite pH is a relatively simple process if the acid (or base) of appropriate dissociation constant is found. Small variations in pH are obtained by variations in the ratio of the acid to the salt concentration according to the equation:
pH=pkₐ+log [salt]/[acid]

The vaginal cavity exhibits an aqueous environment containing secreting glands whose fluids create an acidic pH in the range of 4.5 to 5.5. Therefore, in order to generate a buffer solution having a pH of approximately 4.5, an acid with a pkₐ of approximately this value would be needed. Monoprotic acetic acid, for example, has a pkₐ value of 4.74 and the first two ionizable protons from citric acid have values of 3.13 and 4.76, respectively. Lactic acid is another example with a pkₐ of approximately 3.9.

While theoretical amounts of an acid and salt can be derived from the equation above, in a formulation that is a complicated mixture of many dissolved species it is more practical to titrate a given amount of an acid, typically citric acid or acetic acid, with a solution of known concentration of either sodium or potassium hydroxide until the desired pH value is obtained in the actual formulation.

The resistance of a solution to changes in hydrogen ion concentration upon the addition of small amounts of acid or alkali is termed buffer action. A solution possessing such properties is known as a buffer solution. It is said to possess reserve acidity and reserve alkalinity. Buffer solutions usually consist of solutions containing a mixture of a weak acid and it's sodium or potassium salt or of a weak base and it's salt. A buffer then is usually a mixture of an acid and it's conjugate base.

The unit cells also have an external phase. This phase is lipoidal and is the continuous phase of the systems. The term lipoidal pertains to any of a group of organic compounds comprising neutral fats, fatty acids, waxes, phosphatides, petrolatum, fatty acid esters of monoprotic alcohols and mineral oils having the following common properties: insoluble in water, soluble in alcohol, ether, chloroform or other fat solvents, and which exhibit a greasy feel. Examples of oils suitable for use in the delivery systems are mineral oils with viscosities of 5.6 to 68.7 centistokes, preferably 25 to 65 centistokes, and vegetable oils illustrated by coconut, palm kernel, cocoa butter, cottonseed, peanut, olive, palm, sunflower seed, sesame, corn, safflower, rape seed, soybean and fractionated liquid triglycerides of short chain (naturally derived) fatty acids. This external phase may also contain fragrances, colors, flavors, and buffers.

The active agent may be any of those which are approved for or used for the treatment, prophylaxis, cure or mitigation of any disease of the vagina, urinary tract, cervix or other female reproductive organ or inducement of conception; for aesthetic or cosmetic usage, for diagnostic purposes; for systemic drug therapy; or for sex determination of offspring. The agent must have utility when administered by delivery to all or a portion of the vaginal surfaces. Potential agents are normally well-known due to their need for governmental approval or common usage. At least a portion of the active agent must usually be contained in the internal phase in order to obtain the release characteristics of the systems.

It has been found that when active agents including antimycotics, such as butoconazole, are used as part of the active agent, the conventional treatment period or quantity of agent used is reduced by at least 25%. Normally a controlled release drug system reduces the number of times a day a drug must be administered. However, it does not affect the overall length of treatment. With respect to certain active agents it has been discovered that the drug delivery systems described herein reduces the treatment period by at least 25%. Thus, the treatment of microbes can be achieved in much shorter time or with substantially less drug with the systems of the invention.

Adjacent unit cells have common external phases. The external phase of the unit cells provides the continuous phase of the system. The unit cells may utilize emulsifiers. Preferably, the emulsifiers are soluble in the lipoidal or external phase. Suitable emulsifiers are those oil miscible, surface active compounds which are acceptable for use in foods, pharmaceuticals, and/or cosmetics. Examples of such emulsifiers are low molecular weight polyglycerols, which have been esterified with fatty acids or fatty acid esters, or mono and diglyceride mixtures alone or with the addition of metallic soaps, such as, aluminum stearate. The metallic soaps appear to improve the characteristics of some of the emulsions.

The systems can be introduced into the vaginal cavity by the use of conventional applicators or other coating or spraying means. Although the systems are deformable at physiological temperatures, approximately 37 degrees C., they do not lose integrity in the same manner as the systems of the prior art. The present delivery systems, unlike prior art systems, are not characterized by offensive leakage from the vaginal cavity following the insertion of the system. Since the present systems break down over an extended period, nonaqueous components are either absorbed or released from the vaginal cavity at an unnoticeable rate, which makes no significant increase in normal secretions.

The characteristics of these systems are a result of their inherent integrity under vaginal conditions. The systems release the active agent in the vaginal cavity due to diffusion of the active agent, rupture of the unit cells and/or a combination of these two mechanisms. This release of active agent can be linear or non-linear depending on the composition of the system. Factors which affect the release rate are the percentage of active agent contained in each of the phases; and the type of system, such as, emulsion, double emulsion, suspension; thickness of the external membrane; amount and nature of emulsifier in the external phase; osmotic pressure of the internal phase; pH of the internal phase; diffusibility of the active species through the external phase membrane; etc. Within the physiological environment of the vaginal cavity all of the chemical and physical forces present, including fluids, enzymes, pH, chemical balance, temperature, and shear forces from body movement, affect the rate of breakdown of the system. These forces are not believed to destroy the integrity of the systems at the same rate as other prior art systems.

The systems may be prepared by well-known continuous or batch processes. When processing using conventional emulsions, shear force is applied to the system components by use of homogenizers, mills, impingement surfaces, ultrasound, shaking or vibration. Unlike conventional emulsions, the mixing shear should be at low levels in order to prevent destruction of the system resulting from excess energy used in the process. Temperature is not usually a critical factor in the preparation of the systems. The temperatures utilized will be dependent upon the final end product desired. Phase combination is usually performed at ambient temperatures.

The systems may be prepared by mixing the internal with the external phase in a planetary-type mixer or sweep blade with counter-rotating mixer by pumping the aqueous phase into the oil phase. Another manner of preparing the systems is by use of a continuous mixer, which comprises multiple impellers. The external phase is first introduced into the continuous mixer until it reaches the level of the lowest impeller in the mixing chamber. The two phases are then simultaneously introduced through the bottom of the mixer in proper proportion as its impeller or impellers rotate to apply a shear to the components. The finished product emerges through the top of the mixer. The actual speed of the impeller or impellers will vary, depending upon the product produced, as will the rate of flow of the two phase streams. In some preparations, both methods are used. The emulsion is prepared in the planetary-type or sweep blade with the counter-rotating mixer. The emulsion is then pumped through the continuous mixer to increase emulsion viscosity.

Depending upon the characteristics, such as solubility, etc., of the active pharmaceutically active ingredient, the active ingredient may be added in either the aqueous or oil phase. In either case, the active ingredient may be added into the appropriate phase to preserve its therapeutic nature and activity. Where the active is both water and oil soluble or minimally water and/or oil soluble, the active may be dispersed in the phase resulting in the most physically and chemically stable product or resulting in the cost effective and/or simplified production process.

The following examples are illustrative of preferred embodiments of the invention and are not to be construed as limiting the inventive subject matter thereto. All polymer molecular weights are mean average molecular weights. All percentages are based on the percent by weight of the final delivery system or formulation prepared unless otherwise indicated and all totals equal 100% by weight:

### EXAMPLE 1

This example demonstrates the preparation of a formulation according to the present inventive subject matter.

| | |
|---|---|
| Water, Purified, USP | 37.819 |
| Sorbitol Solution, USP | 39.978 |
| Propylene Glycol, USP | 5.00 |
| Edetate Disodium, USP | 0.050 |
| Butoconazole Nitrate, USP | 2.000 |
| Mineral Oil, USP | 8.032 |
| Polyglyceryl-3-Oleate | 2.713 |
| Glyceryl monoisostearate | 2.713 |
| Microcrystalline Wax, NF | 0.452 |
| Silicon Dioxide, Hydrophobic | 1.013 |
| Methylparaben, NF | 0.180 |
| Propylparaben, NF | 0.050 |

NB: The amount of active ingredient and water to be added is calculated per batch based upon the assay and water content of the raw materials.
General Method of Preparation (Scale-up/Submission Batch)

The water, sorbitol solution and edetate disodium are loaded into a stainless steel mixing tank equipped with a cover and variable speed mixer and mixed at room temperature until all solids are dissolved. At this time after water and sorbitol are mixed if buffers are used i.e., citrate salts or others, they are added to the solution and dissolved. Butoconazole nitrate is added to this solution and mixed until dissolved. The mineral oil, polyglyceryl-3-oleate, glyceryl monoisostearate and microcrystalline wax are loaded into a stainless steel jacketed kettle equipped with a sweep blade and variable speed mixer and mixed at 70-75° C. until all solids are dissolved. Methylparaben and propylparaben are added and dissolved at 70-75° C. While mixing, silicon dioxide is added to the kettle and mixed to create an initial dispersion. While mixing, the material formed is transferred through a colloid mill into a stainless steel jacketed kettle equipped with counter rotation blade and sweep blade. While mixing, the water, sorbitol solution, edetate disodium and butoconazole nitrate mixture is added in a controlled fashion by means of a transfer pump until addition is complete. Mixing is then continued for a predetermined period of time to establish the preliminary emulsion. The preliminary emulsion is then transferred by means of a transfer pump through a secondary mixing chamber at pre-established flow rates and mixing speeds in order to achieve final viscosity. The material is then transferred into bulk containers for packaging into individual applicators.

### EXAMPLE 2

| | |
|---|---|
| Water, Purified, USP | 39.069 |
| Sorbitol Solution, USP | 39.978 |
| Propylene Glycol, USP | 3.75 |
| Edetate Disodium, USP | 0.050 |
| Butoconazole Nitrate, USP | 2.000 |
| Mineral Oil, USP | 8.032 |
| Polyglyceryl-3-Oleate | 2.713 |
| Glyceryl monoisostearate | 2.713 |
| Microcrystalline Wax, NF | 0.452 |
| Silicon Dioxide, Hydrophobic | 1.013 |
| Methylparaben, NF | 0.180 |
| Propylparaben, NF | 0.050 |

NB: The amount of active ingredient and water to be added is calculated per batch based upon the assay and water content of the raw materials.

The formulation was prepared in accordance the general methodology provided herein.

1. A method for the local treatment of a vulvovaginal candidiasis condition diagnosable by a KOH smear test or other fungal speciation test, which comprises:
treating said vulvovaginal candidiasis condition caused by a species of Candida selected from the group consisting of dubliniensis, tropicalis, glabrata, parapsilosis, krusei, and lusitaniae by applying to the vaginal tissue of a human a formulation comprising:
about 35 to about 45% w/w sorbitol solution; about 3 to about 8% w/w propylene glycol; about 0.001 to about 1% w/w edetate disodium; about 5 to about 11% w/w mineral oil; about 0.5 to about 5% w/w polyglyceryl-3-oleate; about 0.5 to about 5% w/w glyceryl monoisostearate; about 0.001 to about 1% w/w microcrystalline wax; about 0.5 to about 2% w/w silicon dioxide; about 0.001 to about 1% w/w methylparaben; about 0.001 to about 1% w/w propylparaben; about 25 to about 45% w/w water; and about 0.5 to about 5% w/w butoconazole nitrate; and
wherein the treatment is a single dose treatment.

2. The method according to claim 1, wherein said formulation further comprises:
about 38 to about 40% w/w sorbitol solution; about 4 to about 6% w/w propylene glycol; about 0.01 to about 0.5% w/w edetate disodium; about 6 to about 9% w/w mineral oil; about 2 to about 3% w/w polyglyceryl-3-oleate; about 2 to about 3% w/w glyceryl monoisostearate; about 0.01 to about 0.8% w/w microcrystalline wax; about 0.09 to about 0.9% w/w silicon dioxide; about 0.01 to about 0.5% w/w methylparaben; about 0.01 to about 0.5% w/w propylparaben; about 30 to about 40% w/w water; and about 1.5 to about 3.5% w/w butoconazole nitrate.

3. The method according to claim 2, wherein said formulation further comprises:
about 39.978% w/w sorbitol solution; about 5% w/w propylene glycol; about 0.05% w/w edetate disodium; about 8.032% w/w mineral oil; about 2.713% w/w polyglyceryl-3-oleate; about 2.713% w/w glyceryl monoisostearate; about 0.452% w/w microcrystalline wax; about 1.013% w/w silicon dioxide; about 0.18% w/w methylparaben; about 0.05% w/w propylparaben; about 37.819% w/w water; and about 2.0% w/w butoconazole nitrate.

4. The method according to claim 3, wherein the species is C. glabrata.

5. The method according to claim 3, wherein the species is C. tropicalis.

6. A method for the treatment of a vaginal fungal condition, which comprises:
administering a single dose composition comprising about 38 to about 40% w/w sorbitol solution; about 4 to about 6% w/w propylene glycol; about 0.01 to about 0.5% w/w edetate disodium; about 6 to about 9% w/w mineral oil; about 2 to about 3% w/w polyglyceryl-3-oleate; about 2 to about 3% w/w glyceryl monoisostearate; about 0.01 to about 0.8% w/w microcrystalline wax; about 0.09 to about 0.9% w/w silicon dioxide; about 0.01 to about 0.5% w/w methylparaben; about 0.01 to about 0.5% w/w propylparaben; about 30 to about 40% w/w water; and about 1.5 to about 3.5% w/w butoconazole nitrate;
wherein the vaginal fungal condition is a vulvovaginal candidiasis condition caused by a Candida species selected from the group consisting of dubliniensis, tropicalis, glabrata, parapsilosis, krusei, and lusitaniae, and
wherein the ratio of polyglyveryl-3-oleate to glyceryl monoisostearate is about 1:0.1-10.

7. The method according to claim 6, wherein the species is C. glabrata.

8. The method according to claim 6, wherein the species is C. tropicalis.

9. A method for the treatment of an unidentified vulvovaginal fungal condition, which comprises:
administration to said fungal condition a bioadhesive, single dose treatment formulation comprising from about 0.500 to about 5.000% w/w butoconazole nitrate; and
wherein the unidentified vulvovaginal fungal condition is caused by a Candida species selected from the group consisting of dubliniensis, tropicalis, glabrata, parapsilosis, krusei, and lusitaniae.

10. The method according to claim 9, wherein said formulation further comprises: about 35 to about 45% w/w sorbitol solution; about 3 to about 8% w/w propylene glycol; about 0.001 to about 1% w/w edetate disodium; about 5 to about 11% w/w mineral oil; about 0.5 to about 5% w/w polyglyceryl-3-oleate; about 0.5 to about 5% w/w glyceryl monoisostearate; about 0.001 to about 1% w/w microcrystalline wax; about 0.5 to about 2% w/w silicon dioxide; about 0.001 to about 1% w/w methylparaben; about 0.001 to about 1% w/w propylparaben; about 25 to about 45% w/w water; and about 0.5 to about 5% w/w butoconazole nitrate.

11. The method according to claim 10, wherein said formulation further comprises:
about 38 to about 40% w/w sorbitol solution; about 4 to about 6% w/w propylene glycol; about 0.01 to about 0.5% w/w edetate disodium; about 6 to about 9% w/w mineral oil; about 2 to about 3% w/w polyglyceryl-3-oleate; about 2 to about 3% w/w glyceryl monoisostearate; about 0.01 to about 0.8% w/w microcrystalline wax; about 0.09 to about 0.9% w/w silicon dioxide; about 0.01 to about 0.5% w/w methylparaben; about 0.01 to about 0.5% w/w propylparaben; about 30 to about 40% w/w water; and about 1.5 to about 3.5% w/w butoconazole nitrate.

12. The method according to claim 10, wherein said formulation further comprises:
about 39.978% w/w sorbitol solution; about 5% w/w propylene glycol; about 0.05% w/w edetate disodium; about 8.032% w/w mineral oil; about 2.713% w/w polyglyceryl-3-oleate; about 2.713% w/w glyceryl monoisostearate; about 0.452% w/w microcrystalline wax; about 1.013% w/w silicon dioxide; about 0.18% w/w methylparaben; about 0.05% w/w propylparaben; about 37.819% w/w water; and about 2.0% w/w butoconazole nitrate.

13. The method according to claim 12, wherein the species is C. glabrata.

14. The method according to claim 12, wherein the species is C. tropicalis.

15. The method according to claim 10, wherein the bioadhesive formulation minimizes leakage from the vaginal cavity of a human.

16. The method according to claim 10, wherein the treatment provides peak plasma levels of the butoconazole nitrate at about 6 to about 48 hours after administration and retains activity for at least 4 days.

17. A method for the treatment of a fungal condition diagnosable by KOH smear test or other fungal speciation test, which comprises:
application to a vulvovaginal candidiasis condition caused by a member selected from the group consisting of Candida dubliniensis, Candida tropicalis, Candida glabrata, Candida parapsilosis, mycelial Candida, Candida krusei, and Candida lusitaniae and mixtures thereof of a treatment comprising:
about 35 to about 45% w/w sorbitol solution; about 3 to about 8% w/w propylene glycol; about 0.001 to about 1% w/w edetate disodium; about 5 to about 11% w/w mineral oil; about 0.5 to about 5% w/w polyglyceryl-3-oleate; about 0.5 to about 5% w/w glyceryl monoisostearate; about 0.001 to about 1% w/w microcrystalline wax; about 0.5 to about 2% w/w silicon dioxide; about 0.001 to about 1% w/w methylparaben; about 0.001 to about 1% w/w propylparaben; about 25 to about 45% w/w water; and about 0.5 to about 5% w/w butoconazole nitrate.

18. The method according to claim 17, wherein the treatment is a single dose treatment.

19. A method for the local treatment of an unidentified vaginal fungal condition comprising:
a single administration of a composition consisting essentially of: about 38 to about 40% w/w sorbitol solution; about 4 to about 6% w/w propylene glycol; about 0.01 to about 0.5% w/w edetate disodium; about 6 to about 9% w/w mineral oil; about 2 to about 3% w/w polyglyceryl-3-oleate; about 2 to about 3% w/w glyceryl monoisostearate; about 0.01 to about 0.8% w/w microcrystalline wax; about 0.09 to about 0.9% w/w silicon dioxide; about 0.01 to about 0.5% w/w methylparaben; about 0.01 to about 0.5% w/w propylparaben; about 30 to about 40% w/w water; and about 1.5 to about 3.5% w/w butoconazole nitrate.; and
wherein the administration is to a vulvovaginal candidiasis condition caused by any member selected from the group consisting of dubliniensis, tropicalis, glabrata, parapsilosis, krusei, and lusitaniae.

20. The method according to claim 19, wherein the species is C. glabrata.

21. The method according to claim 19, wherein the species is C. tropicalis.

22. A method for the treatment of a fungal condition diagnosable by KOH smear test or other fungal speciation, comprising:
treating a candidiasis condition caused by a species selected from the group consisting of dubliniensis, tropicalis, glabrata, parapsilosis, krusei, and lusitaniae by applying to the vaginal tissue a multiphase formulation in a single dose;
wherein the multiphase formulation comprises:
a hydrophilic phase, which comprises: about 38 to about 40% w/w sorbitol solution; about 3 to about 8% w/w propylene glycol; about 0.001 to about 1% w/w edetate disodium; about 25 to about 45% w/w water; and about 0.5 to about 5% w/w butoconazole nitrate; and
a hydrophobic phase which comprises about 5 to about 11% w/w mineral oil; about 0.5 to about 5% w/w polyglyceryl-3-oleate; about 0.5 to about 5% w/w glyceryl monoisostearate; about 0.001 to about 1% w/w microcrystalline wax; about 0.5 to about 2% w/w silicon dioxide; about 0.001 to about 1.000% w/w methylparaben; and about 0.001 to about 1% w/w propylparaben.

23. The method according to claim 22, wherein the hydrophobic phase and hydrophilic stage for a bioadhesive dosage form provides peak plasma levels of butoconazole nitrate at about 6 to about 48 hours and retains activity for at least 4 days.

24. A method for the treatment of an undiagnosable vulvovaginatis condition comprising:
treating a condition caused by a species of Candida selected from the group consisting of dubliniensis, tropicalis, glabrata, parapsilosis, krusei, and lusitaniae by applying to the vaginal tissue a multiphase formulation in a single dose to provide a Candida species kill rate of about 50 to about 100% for a period of at least about 4 days.

25. The method according to claim 24, wherein the multiphase formulation is administered via an applicator device which is designed to apply the formulation evenly over the vaginal tissue of a human.

26. A method according to claim 24, wherein the species is C. glabrata.

27. A method according to claim 24, wherein the species is C. tropicalis.
